(19) United States
(12) Patent Application Publication
(10) Pub. No.: US 2018/0206723 A1
(43) Pub. Date: Jul. 26, 2018

(54) Holographic Operation Equipment, Holographic Control Equipment, Holographic Operation Method and Holographic Control Method

(71) Applicant: BOE Technology Group Co., Ltd., Beijing (CN)

(72) Inventors: Yuxin Zhang, Beijing (CN); Yongda Ma, Beijing (CN); Pan Li, Beijing (CN)

(73) Assignee: BOE Technology Group Co., Ltd., Beijing (CN)

(21) Appl. No.: 15/539,787

(22) PCT Filed: Jan. 3, 2017
(86) PCT No.: PCT/CN2017/000017

§ 371 Date: Jun. 26, 2017

(30) Foreign Application Priority Data
Apr. 14, 2016 (CN) 201610232400.4

(51) Int. Cl. A61B 5/00; A61B 34/35; G03H 1/04; G03H 1/22; G16H 30/20

(52) U.S. Cl. CPC A61B 5/0022; A61B 5/745; A61B 5/0082; A61B 2090/367; G03H 1/0402; G03H 1/2294; G16H 30/20; A61B 34/35

(57) ABSTRACT

Holographic operation equipment, including: a holographic image generating device configured to generate holographic image information including an operated object; an operation device configured to operate the operated object; an image transmitting device configured to transmit the holographic image information; and an information receiving device configured to receive a control signal and send the control signal to the operation device to control the operation of the operation device. The operation error resulted in panel displaying can be reduced. A holographic control equipment, a holographic operation method, a holographic control method and a telemedicine system are further provided.

### TECHNICAL FIELD

Embodiments of the present disclosure relate to holographic operation equipment, holographic control equipment, a holographic operation method, a holographic control method and a telemedicine system.

### BACKGROUND

Currently, telemedicine may perform remote consultation, diagnosis and treatment to the sick and wounded on remote areas, islands or ships with poor medical conditions through Internet on the basis of computer technology, telemetering and telecontrol engineering.

At present, telemedicine technology has developed from the original television monitoring and telephone remote diagnosis to the comprehensive transmission of digit, image and voice by utilization of high-speed network, and achieves the real-time communication by utilization of voice and high-definition (HD) images. However, the current telemedicine technology (e.g., remote surgery) can only flatly display the patient site on a display, and then the doctor can operate surgical instruments according to the patient site on the display. As flatly displayed images cannot fully reflect three-dimensional (3D) information of the patient site such as volume and depth, operation errors can be easily caused, and a slight deviation in surgery is likely to cause harm to the patient and even threaten the life.

### SUMMARY

According to one aspect of this disclosure, a holographic operation equipment is provided, comprising: a holographic image generating device configured to generate holographic image information including an operated object; an operation device configured to operate the operated object; an image transmitting device configured to transmit the holographic image information; and an information receiving device configured to receive a control signal and send the control signal to the operation device to control the operation of the operation device, wherein the control signal is obtained according to the transmitted holographic image information.

For example, in the operation equipment provided in an embodiment of this disclosure, the holographic image generation device includes: a light source component, an optical module, a holographic storage medium and an image acquisition means.

For example, in the operation equipment provided in an embodiment of this disclosure, the light source component can emit a first light beam and a second light beam irradiating the operated object; the second light beam is led to the operated object by the optical module; light reflected or transmitted from the operated object is irradiated to the holographic storage medium after interfering with the first light beam, so as to store the holographic image information in the holographic storage medium; the light source component also emits a third light beam; the third light beam irradiates the holographic storage medium to generate imaging beams emitted from the holographic storage medium; the imaging beams irradiate the image acquisition means; and the image acquisition means generates data corresponding to the holographic image information and sends the data to the image transmitting device.

For example, in the operation equipment provided in an embodiment of this disclosure, the light source component includes a first laser; the optical module includes a light dividing unit; and an initial light beam emitted by the first laser is divided into the first light beam, the second light beam and the third light beam by the light dividing unit.

For example, in the operation equipment provided in an embodiment of this disclosure, the light source component includes a first laser and a second laser; the optical module includes a light dividing unit; an initial light beam emitted by the first laser is divided into the first light beam and the second light beam by the light dividing unit; and the second laser emits the third light beam.

For example, in the operation equipment provided in an embodiment of this disclosure, the holographic storage medium includes photorefractive crystals, photochromic materials or photopolymers.

For example, in the operation equipment provided in an embodiment of this disclosure, the holographic storage medium is disposed on a shifter; and when the holographic operation equipment operates, the shifter allows different positions of the holographic storage medium to face light beams generated after the light reflected or transmitted from the operated object interferes with the first light beam.

For example, in the operation equipment provided in an embodiment of this disclosure, the shifter is a revolving table.

For example, in the operation equipment provided in an embodiment of this disclosure, the emission position of the third light beam may change around the holographic storage medium.

For example, in the operation equipment provided in an embodiment of this disclosure, the image acquisition means includes a charge-coupled device (CCD) or complementary metal oxide semiconductor (CMOS) imaging unit.

For example, in the operation equipment provided in an embodiment of this disclosure, the operation device includes a mechanical arm; and surgical instruments may be disposed on the mechanical arm.

For example, in the operation equipment provided in an embodiment of this disclosure, the image transmitting device and the information receiving device are respectively in signal connection with an external device via Internet.

According to another aspect of this disclosure, a holographic operation method is provided, comprising: generating holographic image information including an operated object; transmitting the holographic image information to an external device; receiving a control signal from the external device, in which the control signal is obtained according to the transmitted holographic image information; and controlling the operation on the operated object according to the control signal.

For example, in the operation method provided in an embodiment of this disclosure, the step of generating the holographic image information including the operated object includes: generating a first light beam and a second light beam irradiating the operated object; leading the second light beam to the operated object; irradiating light reflected or transmitted from the operated object to a holographic storage medium after the light interferes with the first light beam, so as to store the holographic image information in the holographic storage medium; generating a third light beam to irradiate the holographic storage medium to generate imaging beams emitted from the holographic storage medium; and irradiating the imaging beams to an image acquisition means to generate data corresponding to the holographic image information.

For example, in the operation method provided in an embodiment of this disclosure, an initial light beam emitted by a first laser is divided into the first light beam, the second light beam and the third light beam.

For example, in the operation method provided in an embodiment of this disclosure, an initial light beam emitted by a first laser is divided into the first light beam and the second light beam; and a second laser is adopted to emit the third light beam.

For example, in the operation method provided in an embodiment of this disclosure, the holographic storage medium includes photorefractive crystals, photochromic materials or photopolymers.

For example, in the operation method provided in an embodiment of this disclosure, different positions of the holographic storage medium are allowed to face light beams generated after the light reflected or transmitted from the operated object interferes with the first light beam.

For example, in the operation method provided in an embodiment of this disclosure, the emission position of the third light beam may change around the holographic storage medium.

For example, in the operation method provided in an embodiment of this disclosure, the step of controlling the operation on the operated object according to the control signal includes: controlling the movement of a mechanical arm on an operation device according to the control signal; and allowing the mechanical arm to operate the operated object during the movement of the mechanical arm.

For example, in the operation method provided in an embodiment of this disclosure, the image transmitting and information receiving step includes: transmitting the holographic image information to an external device via Internet; and receiving the control signal from the external device via Internet.

According to another aspect of this disclosure, a holographic control equipment is provided, comprising: an image receiving device configured to receive holographic image information; a holographic image reproduction device configured to reproduce a holographic image according to the holographic image information; an operation control device configured to generate an operation control signal; and an information output device configured to receive the operation control signal and output the operation control signal, wherein the operation control signal is generated according to the holographic image.

For example, in the holographic control equipment provided in an embodiment of this disclosure, the image reproduction device includes: a light source configured to emit reproduced beams; a spatial light modulator configured to receive the holographic image information and convert the holographic image information into optical signals when irradiated by the reproduced beams; and an imaging unit configured to reveal the optical signals as the holographic image.

For example, in the holographic control equipment provided in an embodiment of this disclosure, the light source includes lasers.

For example, in the holographic control equipment provided in an embodiment of this disclosure, the spatial light modulator includes a liquid crystal light valve, a micro-electro-mechanical system (MEMS) spatial light modulator, a digital micromirror device (DMD), photorefractive crystals or an acousto-optic modulator (AOM).

For example, in the holographic control equipment provided in an embodiment of this disclosure, the operation control device includes a touch element, an operating arm or a somatosensory control element.

According to another aspect of this disclosure, a holographic control method is provided, comprising: receiving holographic image information; reproducing a holographic image according to the holographic image information; generating an operation control signal; and receiving the operation control signal and outputting the operation control signal, wherein the operation control signal is generated according to the holographic image.

For example, in the holographic control method provided in an embodiment of this disclosure, the step of reproducing the holographic image according to the holographic image information includes: emitting reproduced beams to a spatial light modulator; allowing the spatial light modulator to receive the holographic image information and convert the holographic image information into optical signals when irradiated by the reproduced beams; and revealing the optical signals as the holographic image.

For example, in the holographic control method provided in an embodiment of this disclosure, the step of emitting the reproduced beams includes: adopting a laser to obtain the reproduced beams.

For example, in the holographic control method provided in an embodiment of this disclosure, the spatial light modulator includes a liquid crystal light valve, an MEMS spatial light modulator, a DMD, photorefractive crystals or an AOM.

For example, in the holographic control method provided in an embodiment of this disclosure, the step of generating the operation control signal includes: generating the operation control signal by operating a touch element, an operating arm or a somatosensory control element.

According to another aspect of this disclosure, a telemedicine system is provided, comprising the holographic operation equipment in any embodiments of this disclosure and the holographic control equipment in any embodiments of this disclosure.

### BRIEF DESCRIPTION OF THE DRAWINGS

Simple description will be given below to the accompanying drawings of the embodiments to provide a more clear understanding of the technical proposals of the embodiments of the present disclosure. Obviously, the drawings described below only involve some embodiments of the present disclosure but are not intended to limit the present disclosure.

FIG. 1 is a schematic block diagram of holotherapy equipment;

FIG. 2 is a schematic structural view of the holotherapy equipment;

FIG. 3 is a flow diagram of a holographic operation method;

FIG. 4 is a schematic block diagram of holotherapy control equipment;

FIG. 5 is a schematic structural view of the holotherapy control equipment;

FIG. 6 is a flow diagram of a holotherapy control method;

FIG. 7 is a schematic block diagram of a telemedicine system; and

FIG. 8 is a schematic structural view of the telemedicine system.

### DETAILED DESCRIPTION

In order to make objects, technical details and advantages of the embodiments of the invention apparent, the technical solutions of the embodiments will be described in a clearly and fully understandable way in connection with the drawings related to the embodiments of the invention. Apparently, the described embodiments are just a part but not all of the embodiments of the invention. Based on the described embodiments herein, those skilled in the art can obtain other embodiment(s), without any inventive work, which should be within the scope of the invention.

Unless otherwise defined, all the technical and scientific terms used herein have the same meanings as commonly understood by one of ordinary skill in the art to which the present invention belongs. The terms “first,” “second,” etc., which are used in the description and the claims of the present application for invention, are not intended to indicate any sequence, amount or importance, but distinguish various components. Also, the terms such as “a,” “an,” etc., are not intended to limit the amount, but indicate the existence of at least one. The terms “comprise,” “comprising,” “include,” “including,” etc., are intended to specify that the elements or the objects stated before these terms encompass the elements or the objects and equivalents thereof listed after these terms, but do not preclude the other elements or objects. The phrases “connect”, “connected”, etc., are not intended to define a physical connection or mechanical connection, but may include an electrical connection, directly or indirectly.

Embodiments of the present disclosure provide holographic operation equipment, holographic control equipment, a holographic operation method, a holographic control method and a telemedicine system, for instance, holotherapy equipment, holotherapy control equipment, a holographic operation method and a holotherapy control method applied in the medical field. Obviously, the embodiment of the present disclosure may be not limited to be applied in the medical field and may also be applied in other fields such as manufacturing, mining, agriculture and animal husbandry, and corresponding operated objects may be manufactured products, excavated minerals, planted crops, fed livestock, etc. Non-limiting description will be given below by taking the medical field as an example, wherein the holotherapy equipment is applied to the patient end; the holotherapy control equipment is applied to the doctor end; and the doctor may control the holotherapy equipment through the holotherapy control equipment, and hence perform medical operation on the patient. The holographic operation method is an operation method corresponding to the holotherapy equipment. The holotherapy control method is a control (operation) method corresponding to the holotherapy control equipment. The telemedicine system comprises the holotherapy equipment and the holotherapy control equipment. Description will be given below to the equipment, the system and the method.

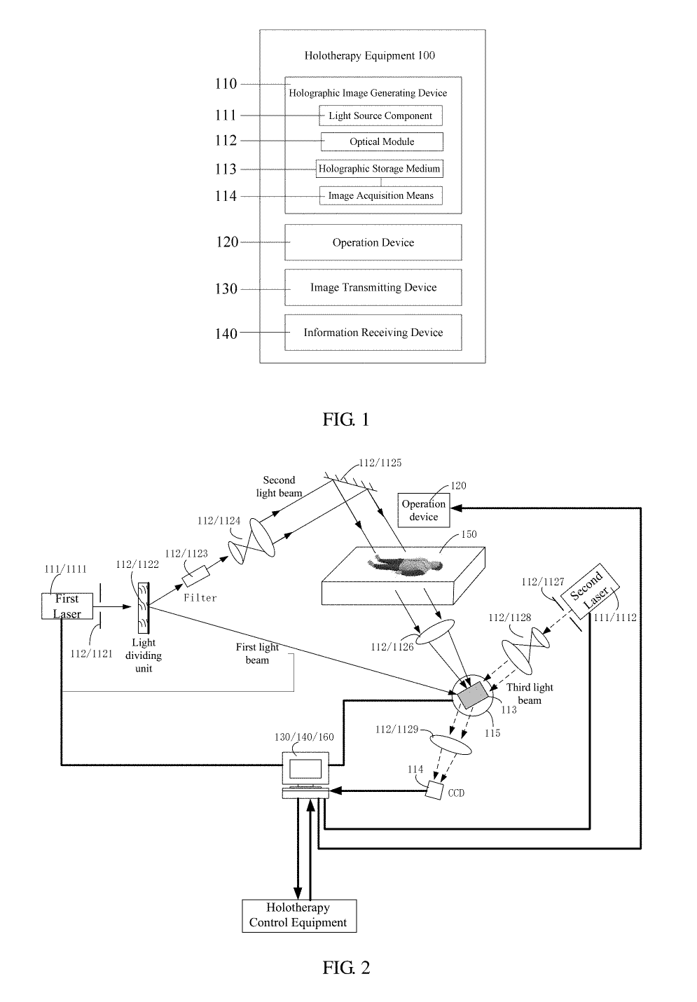

The first embodiment of the present disclosure provides holotherapy equipment. Description will be given below to the holotherapy equipment provided by the first embodiment of the present disclosure with reference to FIGS. 1 and 2. FIG. 1 is a schematic block diagram of the holotherapy equipment provided by the first embodiment of the present disclosure, and FIG. 2 is a schematic structural view of the holotherapy equipment.

As shown in FIG. 1, the holotherapy equipment 100 comprises: a holographic image generating device 110, an operation device 120, an image transmitting device 130 and an information receiving device 140. The holographic image generating device 110 is configured to generate holographic image information including an operated object. The operation device 120 is configured to operate the operated object. The image transmitting device 130 is configured to transmit the holographic image information. The information receiving device 140 is configured to receive a control signal and send the control signal to the operation device 120 so as to control the operation of the operation device 120. The control signal is obtained according to the transmitted holographic image information.

The holographic image generating device 110 is configured to generate the holographic image information including the operated object. In the embodiment, the operated object is the patient or the like, so, for instance, holographic image information of the patient or the patient site is generated. As shown in FIG. 1, the holographic image generating device 110 may include: a light source component 111, an optical module 112, a holographic storage medium 113 and an image acquisition means 114.

As shown in FIG. 2, the light source component 111 is configured to emit light and is relevant light sources capable of realizing holographic record, e.g., laser, infrared light, near infrared light, white light, etc. The light source component 111, for instance, may be implemented by one or more lasers, one or more infrared emitters or a combination thereof. In addition, the light source component 111 may also be other relevant light sources capable of realizing holographic record, e.g., white light sources. Optionally, the light source component 111 may be implemented by a near infrared tunable fiber laser and an infrared transmitting tube. In the infrared transmitting tube, a luminous body is formed by an infrared light-emitting diode (LED) matrix. In an infrared LED, a PN junction is made from materials with high infrared radiation efficiency, and external forward bias injects current into the PN junction to excite infrared light.

According to one example of the present disclosure, the light source component 111 may emit a first light beam and a second light beam, in which the second light beam irradiates the operated object, for instance, the patient. The second light beam may directly irradiate the patient and may also be led to the operated object by the optical module 112. Thus, light reflected or transmitted from the operated object is irradiated to the holographic storage medium 113 after interfering with the first light beam, so as to store holographic image information of the operated object in the holographic storage medium 113.

According to another example of the present disclosure, the light source component 111 not only can emit the first light beam and the second light beam but also can emit a third light beam. The third light beam is configured to irradiate the holographic storage medium to produce imaging beams emitted from the holographic storage medium, and the imaging beams are irradiated to the image acquisition means 114 to form an image.

Alternatively, the light source component 111 includes a first laser 1111 and a second laser 1112. The first laser 1111 is configured to emit the first light beam and the second light beam. The second laser 1112 is configured to emit the third light beam. For instance, a light beam emitted by the first laser 1111 is divided into the first light beam and the second light beam by a light dividing unit. Of course, optionally, the light source component 111 may also include three lasers which respectively emit the first light beam, the second light beam and the third light beam.

Alternatively, the light source component may also only include the first laser 1111, and an initial light beam emitted by the first laser 1111 is divided into the first light beam, the second light beam and the third light beam by the light dividing unit.

The optical module 112 is configured to perform operations such as light leading, light splitting and filtering in the holotherapy equipment. For instance, as shown in FIG. 2, the optical module 112 may include a light dividing unit 1122 (e.g., an optical splitter) which is configured to divide the light beam emitted by the first laser 1111 in the optical module 111 into the first light beam and the second light beam.

Optionally, in order to filter off light with certain waveband in the first light beam, the optical module 112 may also include a filter which is configured to filter the light. For instance, a filter 1123 in FIG. 2 is configured to filter the first light beam.

Optionally, in order to change the diameter and the divergence angle of the light beam, the optical module 112 may also include a beam expander. In order to couple the light into a light receiving element under maximal efficiency, the optical module 112 may also include a collimator. For instance, a first beam-expanding collimator 1124 and a second beam-expanding collimator 1128 in FIG. 2 can achieve the function of beam expansion and collimation.

Optionally, in order to adjust a light path so that the light beam can be led to the operated object after divergence or convergence, the optical module 112 may also include one or more lenses, reflectors or plane mirrors or any combination thereof according to the guiding requirement of the light path. For instance, a reflector 1125, a first lens 1126 and a second lens 1129 in FIG. 2 are respectively configured to achieve the function of light reflection or transmission.

Optionally, in order to effectively determine the spectral band width and the intensity of outgoing beams, the optical module 112 may also adopt slits on a light-emitting end of the light source component 111, so as to set adequate gaps. The maximum width of the slit may be 2 mm. The slit is a main part of a spectrometer, and the slits adapted to the light source component 111 may be designed through the spectrometer. For instance, as shown in FIG. 2, a first slit 1121 and a second slit 1127 may be respectively disposed on light-emitting ends of the first laser 1111 and the second laser 1112.

It should be noted that the number of the optical modules 112, for instance, optical elements such as a lens assembly and reflectors, may be increased or reduced in the light path of the first light beam, the second light beam or the third light beam according to actual application demands, so as to realize the adjustment of, for instance, the light direction or the divergence angle.

The holographic storage medium 113 is configured to store optical information. As shown in FIG. 2, in one example of the present disclosure, the holographic storage medium 113 stores interference information of the first light beam and the second light beam. The holographic storage medium 113 may include photorefractive crystals, photochromic materials, photopolymers or the like, wherein the photorefractive crystals store holographic images according to the photorefractive effect, that is, when irradiated by non-uniform light intensity, the change of the local refractive index of the photorefractive crystals is in direct proportion to the incident light intensity. The photorefractive crystals have the advantages of large dynamic range, long storage durability, capability of being fixed, mature growth process, etc. The photorefractive crystals are, for instance, iron-doped lithium niobate crystals (KiNbO3: Fe), barium strontium niobate (SNB), barium titanate (BaTiO3) or the like; and the photopolymers are, for instance, PMMA: DTNB: C60, PQ/PMMA, etc.

The image acquisition means 114 is configured to generate data corresponding to the holographic image information, for instance, converting light into electrical signals. As shown in FIG. 2, in one example of the embodiment of the present disclosure, the image acquisition means 114 converts the third light beam transmitted from the holographic storage medium 113 into electrical information. The image acquisition means 114, for instance, may be implemented by a charge-coupled device (CCD) or complementary metal-oxide semiconductor (CMOS) imaging unit. Both CCD and CMOS can sense light and convert optical signals into digital signals.

As shown in FIG. 2, according to one example of the present disclosure, the image generating process of the holographic image generating device 110 may be as follows. The light source component 111 emits the first light beam and the second light beam for irradiating an operated object 150; the second light beam is led to the operated object 150 by the optical module 112 and irradiates the operated object 150; and light reflected or transmitted from the operated object 150 is irradiated to the holographic storage medium 113 after interfering with the first light beam, so as to store holographic image information in the holographic storage medium.

In addition, the light source component 111 may also emit the third light beam. The third light beam irradiates the holographic storage medium 113 to generate imaging beams emitted from the holographic storage medium 113; the imaging beams are irradiated to the image acquisition means 114; the image acquisition means 114 generates electrical signal data corresponding to the holographic image information on the basis of the imaging beams; and subsequently, the data may be sent to the image transmitting device 130.

According to another example of the present disclosure, the image generating process of the holographic image generating device 110, for instance, may be as follows. As shown in FIG. 2, light emitted by the first laser 1111 is divided into the first light beam and the second light beam by the light dividing unit 1122 after running through the first slit 1121. The first light beam may be taken as a reference beam and the second light beam may be taken as an object beam. The second light beam is filtered by the filter 1123 and subjected to beam expansion and collimation by the first beam-expanding collimator 1124, and subsequently is reflected by the first reflector 1125 to the operated object 150 (e.g., the patient) to produce, for instance, diffuse reflection. Subsequently, light reflected or transmitted from the operated object 150 is converged through the first lens 1126 and irradiated to the holographic storage medium 113. Meanwhile, the first light beam is directly incident or led into the holographic storage medium 113. The first light beam and the second light bream are superimposed to produce interference, and interference information is stored into the holographic storage medium 113.

In addition, in order to read out the optical information in the holographic storage medium 113, light may be emitted by the second laser 1112, is subjected to beam expansion and collimation by the second slit 1127 and the second beam-expanding collimator 1128, and is hence irradiated to the holographic storage medium 113. Light emitted from the holographic storage medium 113 is incident into the image acquisition means 114 after running through the second lens 1129, so that information stored in the holographic storage medium 113 can be converted into electrical signals and read out.

In order to reduce the crosstalk between a plurality of stored holographic images, after writing in one holographic image, the holographic storage medium 113 rotates an angle and then writes in the next holographic image. According to one example of the present disclosure, the holographic storage medium 113 may be disposed on a shifter 115. When the holotherapy equipment 100 operates, the shifter 115 can move the holographic storage medium 113, so that the holographic storage medium can face light beams, produced after the light reflected or transmitted from the operated object 150 interferes with the first light beam, at different angles and different positions.

Alternatively, the emission position of the third light beam may change around the holographic storage medium 113. For instance, the second laser 1112 is disposed on a mobile optical platform which can drive the second laser 1112 to move around the holographic storage medium 113, so that the emission position of the third light beam can change around the holographic storage medium 113.

Optionally, the shifter 115 is a revolving table. The revolving table is, for instance, a single-axis table, a double-axis table or a multi-axis table with three or more than three axes. The multi-axis table is beneficial to improve the pointing accuracy of the revolving table and the holographic storage medium 113 disposed on the revolving table, and is conducive to the storage and read of the holographic images.

In the holotherapy equipment 100 provided by one embodiment of the present disclosure, the operation device 120 is configured to operate the operated object, for instance, perform diagnosis or surgery on the patient. The operation device 120, for instance, includes a mechanical arm, and surgical instruments may be disposed on the mechanical arm. The mechanical arm may perform multi-axis movement in a 3D space. The surgical instruments, for instance, include anaesthetic apparatuses, surgical knifes, vessel clamps, sign monitoring devices, etc. Optionally, the holotherapy equipment 100 may also comprise a first control device 160. The first control device 160 is, for instance, configured to control the operation of the first laser 1111, the second laser 1112 and the shifter 115.

In addition, the image transmitting device 130 is configured to transmit holographic image information to other devices. For instance, as shown in FIG. 2, after receiving the holographic image information including the operated object, sent by the image generating device 110, the image transmitting device 130 sends out the holographic image information, for instance, to the holotherapy control equipment, or stores the holographic image information.

In addition, the information receiving device 140 is configured to receive a control signal obtained according to the transmitted holographic image information, and send the control signal to the operation device 120 to control the operation of the operation device 120. For instance, in FIG. 2, the information receiving device 140 may receive a control signal sent by the holotherapy control equipment.

The information receiving device 140 and the image transmitting device 130 may be in signal connection with an external device via Internet. The Internet, for instance, includes wired network, wireless network or a combination thereof. The external device is, for instance, the holotherapy control equipment.

Optionally, the functions of the image transmitting device 130, the information receiving device 140 and the first control device 160 may be respectively realized by one or more computers. The computer may be a general-purpose computing device or a special-purpose computing device.

According to one example of the present disclosure, the working process of the holotherapy equipment 100 may be as follows. The holographic image generating device 110 generates the holographic image information including the operated object, for instance, generating holographic images of the patient or the patient site. Subsequently, the holographic image information is transmitted to the image transmitting device 130. After receiving the holographic image information, the image transmitting device 130 transmits the holographic image information to an external device, for instance, to the holotherapy control equipment as shown in FIG. 2. After receiving the holographic image information, the holotherapy control equipment generates a control signal, for instance, a surgical operation control signal to the patient, according to the information, and sends the control signal to the holotherapy equipment 100. The information receiving device 140 of the holotherapy equipment 100 receives the control signal and sends the control signal to the operation device 120, and the operation device 120 operates the operated object, for instance, performs surgery on the patient, according to the control signal.

The holotherapy equipment provided by the embodiment of the present disclosure generates the holographic image of the operated object, sends the holographic image to the holotherapy control equipment, and performs medical diagnosis or surgery on the operated object according to the control signal sent by the holotherapy control equipment. As the control signal is generated according to the holographic image of the operated object, the control signal is equivalent to a control instruction issued by a doctor at the scene, so that the medical operation can be more accurate. Thus, the operation error can be greatly reduced, and meanwhile, the medical efficiency can be also improved.

The holotherapy equipment provided by the first embodiment of the present disclosure has been described above. Description will be given below to a holographic operation method provided by the second embodiment of the present disclosure. The holographic operation method is an operation method corresponding to the holotherapy equipment. Only simple description will be given below for the simplicity of the Description.

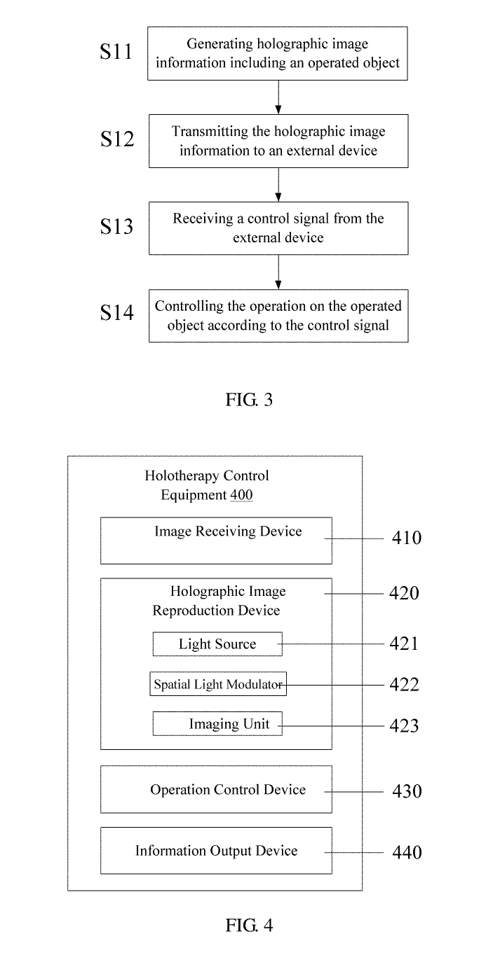

As illustrated in FIG. 3, the holographic operation method 300 comprises the following steps.

S11: generating holographic image information including an operated object. According to one example of the present disclosure, the process of generating the holographic image information is as follows: firstly, generating a first light beam and a second light beam irradiating the operated object; secondly, leading the second light beam to the operated object; and thirdly, irradiating light reflected or transmitted from the operated object to a holographic storage medium after the light interferes with the first light beam, so as to store the holographic image information in the holographic storage medium. Meanwhile, a third light beam may also be generated to irradiate the holographic storage medium to generate imaging beams emitted from the holographic storage medium. Subsequently, the imaging beams are irradiated to an image acquisition means to generate data corresponding to the holographic image information.

Optionally, an initial light beam may be emitted by a first laser and divided into a first light beam, a second light beam and a third light beam.

Optionally, the initial light beam emitted by the first laser may also be divided into the first light beam and the second light beam, and a second laser is adopted to emit the third light beam.

Optionally, the holographic storage medium may be made from materials such as photorefractive crystals, photochromic materials and photopolymers.

Optionally, different positions of the holographic storage medium may be allowed to face light beams produced after the light reflected or transmitted from the operated object interferes with the first light beam, so that a plurality of images can be written into the holographic storage medium, and the crosstalk between the plurality of stored holographic images can be reduced. For instance, after writing in one holographic image, the holographic storage medium 113 rotates an angle and then writes in the next holographic image. Alternatively, a light source component 111 may also be moved. For instance, a first laser 1111 or a second laser 1112 is moved, or a light path is changed by a light guide component, so that the emission position of the third light beam can change around the holographic storage medium 113.

According to one example of the present disclosure, as shown in FIGS. 2 and 3, the process of generating the holographic image may be as follows. Firstly, light emitted by the first laser 1111 is divided into a first light beam and a second light beam by a light dividing unit 1122 after running through a first slit 1121. The first light beam may be taken as a reference beam, and the second light beam may be taken as an object beam. Secondly, the second light beam is filtered by a filter 1123 and subjected to beam expansion and collimation by a first beam-expanding collimator 1124, and then reflected to an operated object 150 (e.g., the patient) by a first reflector 1125 to produce, for instance, diffuse reflection. Thirdly, light reflected or transmitted from the operated object 150 is converged to the holographic storage medium 113 through the first lens 1126. Meanwhile, the first light beam is directly irradiated or led into the holographic storage medium 113. The first light beam and the second light beam are superimposed to produce interference, and interference information is stored into the holographic storage medium 113.

In addition, in order to read out optical information in the holographic storage medium 113, light may be emitted by the second laser 1112, is subjected to beam expansion and collimation by a second slit 1127 and a second beam-expanding collimator 1128, and is hence irradiated to the holographic storage medium 113. Light emitted from the holographic storage medium 113 is incident into an image acquisition means 114 after running through a second lens 1129, so that information stored in the holographic storage medium 113 can be converted into electrical signals and read out.

S12: transmitting the holographic image information to an external device. S13: receiving a control signal from the external device, in which the control signal is obtained according to the transmitted holographic image information. According to one example of the present disclosure, after the holographic image information is generated in the step S11, the holographic image information is transmitted to the external device via Internet in the step S12. For instance, the image information is transmitted to the external device through a communication medium such as a fiber network or a cordless communication network. For instance, as shown in FIG. 2, the image information is transmitted to holotherapy control equipment. Subsequently, the holotherapy control equipment will generate a control signal, for instance, a control signal to perform surgical operation on the patient, according to the received holographic image information, and transmit the control signal via Internet. At this point, in the step S13, the holotherapy equipment may receive the control signal from the external device.

S14: controlling the operation on the operated object according to the control signal. According to one example of the present disclosure, when receiving the control signal from the external device, the holotherapy equipment may control the operation on the operated object according to the control signal, for instance, controlling the movement of a mechanical arm on an operation device 120 according to the control signal. During the movement of the mechanical arm, surgical instruments disposed on the mechanical arm may operate the operated object (for instance, the patient).

The holographic operation method provided by the embodiment of the present disclosure transmits the generated holographic image including the operated object to the external device, receives the control signal generated according to the transmitted holographic image from the external device, and operates the operated object according to the control signal. Thus, the generated control signal can be more accurate, so that the accuracy and the efficiency of medical operations can be greatly improved.

Description has been given above to the holotherapy equipment provided by the first embodiment of the present disclosure and the holographic operation method provided by the second embodiment. The equipment and the method may be applied to the patient end receiving medical care. Further description will be given below to holotherapy control equipment provided by the third embodiment of the present disclosure and a holotherapy control method provided by the fourth embodiment of the present disclosure. The holotherapy control equipment and the holotherapy control method may be applied to the doctor end providing medical services and configured to control the operations of devices on the patient end.

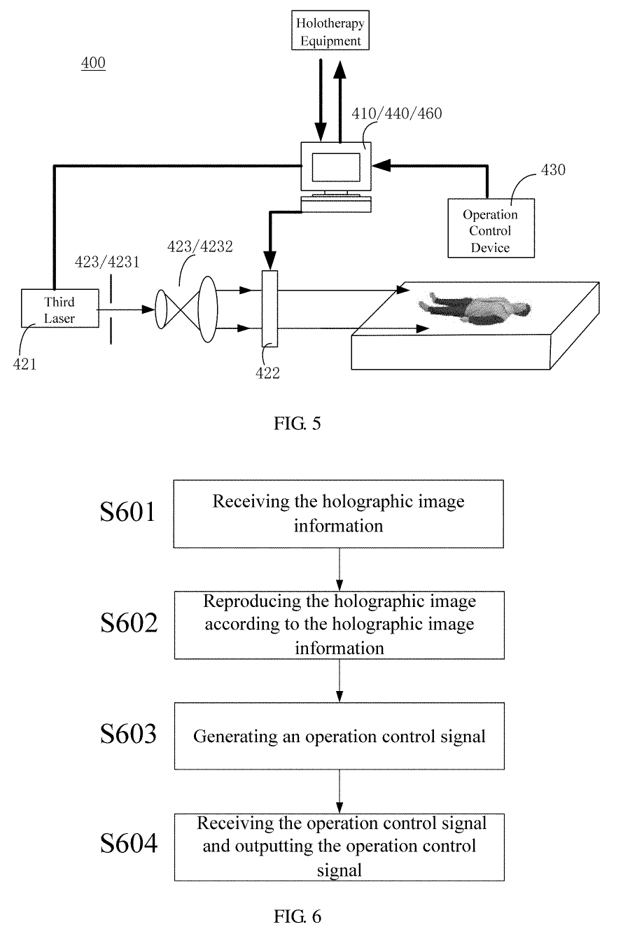

Description will be given below to the holotherapy control equipment provided by the fourth embodiment of the present disclosure with reference to FIGS. 4 and 5. FIG. 4 is a block diagram of the holotherapy control equipment 400 provided by the third embodiment of the present disclosure, and FIG. 5 is a schematic structural view of the holotherapy control equipment 400.

As illustrated in FIG. 4, the holotherapy control equipment 400 comprises: an image receiving device 410, a holographic image reproduction device 420, an operation control device 430 and an information output device 440.

The image receiving device 410 is configured to receive holographic image information. The image receiving device 410, for instance, may be an electronic device communicated with an external device, e.g., a computer, a tablet PC, a notebook computer and a mobile terminal. As shown in FIG. 5, the device may receive the holographic image information including the operated object from the holotherapy equipment via wired or wireless network.

The holographic image reproduction device 420 is configured to reproduce the holographic image according to the holographic image information. The holographic image reproduction device converts the holographic image information received by the image receiving device 410 into a holographic image capable of being viewed by the human eyes. As shown in FIG. 4, in one example of the present disclosure, the holographic image reproduction device 420 includes: a light source 421, a spatial light modulator 422 and an imaging unit 423.

The light source 421 is configured to emit reproduced beams. The light source, for instance, may be a laser or an infrared emitter and may also be other light sources capable of realizing holographic reproduction, for instance, a white light source. As shown in FIG. 5, the light source 421 is, for instance, a third laser 4211.

The spatial light modulator 422 is configured to receive the holographic image information, and can convert the holographic image information into optical signals when irradiated by the reproduced beams. For instance, the spatial light modulator 422 may be a liquid crystal light valve or a micro-electro-mechanical system (MEMS) spatial light modulator, used for holographic reproduction, and may also be a digital micromirror device (DMD), photorefractive crystals, an acousto-optic modulator (AOM), etc.

The imaging unit 423 is configured to reveal the optical signals as the holographic image. The imaging unit 423 may be cooperated with the light source 421 and the spatial light modulator 422 and images the optical signals into the holographic image capable of being viewed by the human eyes through an optical element such as a lens and a reflector. For instance, as shown in FIG. 5, the imaging unit 423, for instance, includes a third slit 4231 and a third beam-expanding collimator 4232. The third slit 4231 may be implemented by utilization of the function of a spectrometer. The third beam-expanding collimator 4232 may be implemented by a beam expanding lens, a collimator or a combination thereof.

According to one example of the present disclosure, in the holographic reproduction device 420, reproduced beams emitted by a first laser 4211 are irradiated to the spatial light modulator 422 after being subjected to beam expansion and collimation through the third slit 4231 and the third beam-expanding collimator 4232, so as to reveal an image of the operated object. For instance, a platform may be arranged, and the image of the operated object is displayed on the platform. In this case, when the doctor sees the holographic image on the platform, it is just as seeing the patient lying on the bed. The doctor can accurately determine how to operate according to the holographic image of the patient, and generates corresponding control signal. Thus, this means facilitates the doctor's medical program formulation and also improves the medical accuracy.

The operation control device 430 is configured to generate an operation control signal. The operator (for instance, the doctor) operates by adoption of the operation control device 430 according to the image of the operated object after determining the medical program and viewing the holographic image of the operated object. For instance, in one example of the present disclosure, the operation control device 430 includes a touch element, an operating arm, a somatosensory control element, etc. After the operator operates the operation control device 430, the operation control device 430 will generate a control signal. Alternatively, the operator may also input operation control information by adoption of an input device of the holotherapy control equipment 400 according to the viewed holographic image information of the operated object, so as to generate an operation control signal. Subsequently, the operation control device 430 sends the control signal of the operation information to an information output device 440.

The information output device 440 receives the operation control signal from the operation control device 430 and outputs the operation control signal to other devices. For instance, as shown in FIG. 5, the operation control signal is outputted to the holotherapy equipment via wired or wireless network.

In addition, the holotherapy control equipment 400 may further comprise a second control device 460. The second control device 460 is configured to control the image reproduction of the image reproduction device 420, for instance, control the light emission of the third laser 4211 and the signal processing of the spatial light modulator 422.

Optionally, as shown in FIG. 5, the image receiving device 410, the information output device 440 and the second control device 460 may be implemented together by one computer or respectively implemented by a plurality of computers. The computer may be a general-purpose computing device or a special-purpose computing device.

The holotherapy control equipment provided by the embodiment of the present disclosure receives the holographic image of the operated object and reproduces the image, so that the doctor can accurately determine the medical program according to the holographic image of the patient, and generate corresponding control signal according to the holographic image. As the holographic image may comprehensively reflect the image of the operated object, when the doctor operates the image, it is just as performing operation on the patient. Thus, this means facilitates the doctor's medical program formulation and also improves the medical accuracy.

The holotherapy control equipment provided by the third embodiment of the present disclosure has been described above. Further description will be given below to the holotherapy control method provided by the fourth embodiment of the present disclosure. The holotherapy control method is a method corresponding to the holotherapy control equipment. Only simple description will be given below for the simplicity of the Description.

Description will be given below to the holotherapy control method provided by the fourth embodiment of the present disclosure with reference to FIG. 6 and FIGS. 4-5. FIG. 6 is a flow diagram of the holotherapy control method provided by the embodiment of the present disclosure.

As shown in FIG. 6, S601: receiving the holographic image information. For instance, the holographic image information including the operated object may be received from the holotherapy equipment via wired or wireless network by utilization of an electronic device such as a computer, a tablet PC, a notebook computer and a mobile terminal.

S602: reproducing the holographic image according to the holographic image information. That is to say, the holographic image reproduction device 420 converts the holographic image information received by the image receiving device 410 into a holographic image capable of being viewed by the human eyes. According to one example of the present disclosure, reproduced beams are emitted to the spatial light modulator 422 by the first laser 4211. After receiving the holographic image information, the spatial light modulator 422 converts the holographic image information into optical signals when irradiated by the reproduced beams, and reveals the optical signals as the holographic image. For instance, a platform may be arranged, and the image of the operated object is displayed on the platform. In this case, when the doctor sees the holographic image on the platform, it is just as seeing the patient lying on the bed. The doctor can accurately determine how to operate according to the holographic image of the patient, and generates corresponding control signal.

According to another example of the present disclosure, the reproduced beams emitted by the first laser 4211 may also be irradiated to the spatial light modulator 422 after preprocessing. For instance, as shown in FIG. 5, the light beams emitted by the first laser 4211 are irradiated to the spatial light modulator 422 after being subjected to divergence angle adjustment through the third slit 4231 and subjected to beam-expanding and collimation through the third beam-expanding collimator 4232, so that the image of the operated image can be reproduced.

Optionally, a liquid crystal light valve, an MEMS spatial light modulator or the like may be adopted as the spatial light modulator to receive the holographic image information and convert the holographic image information into optical signals when irradiated by the reproduced beams.

S603: generating an operation control signal. For instance, the operation control signal is generated by operating a touch element, an operating arm, a somatosensory control element, etc. Alternatively, an input device of the holotherapy control equipment may also be directly adopted to input the operation control signal.

S604: receiving the operation control signal and outputting the operation control signal. For instance, as shown in FIG. 5, the operation control signal generated in the step S603 is received via wired or wireless network, and the control signal is outputted to the holotherapy equipment. In addition, the second control device 460 of the holotherapy control equipment 400 may also be adopted to control the image reproduction of the image reproduction device 420, for instance, control the light emission of the third laser 4211 and the signal processing of the spatial light modulator 422.

The holotherapy control method provided by the embodiment of the present disclosure receives the holographic image of the operated object and reproduces the image, so that the doctor can accurately determine the medical program according to the holographic image of the patient, and generate corresponding control signal according to the holographic image. As the holographic image may comprehensively reflect the image of the operated object, when the doctor operates the image, it is just as performing operation on the patient. Thus, this means facilitates the doctor's medical program formulation and also improves the medical accuracy.

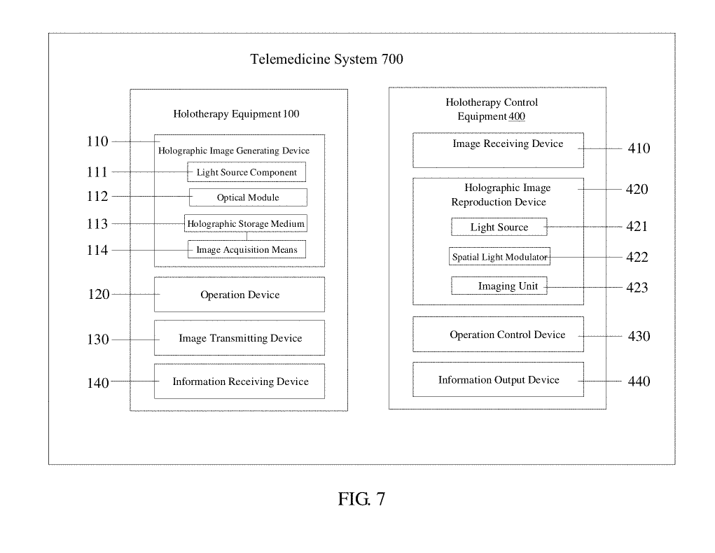

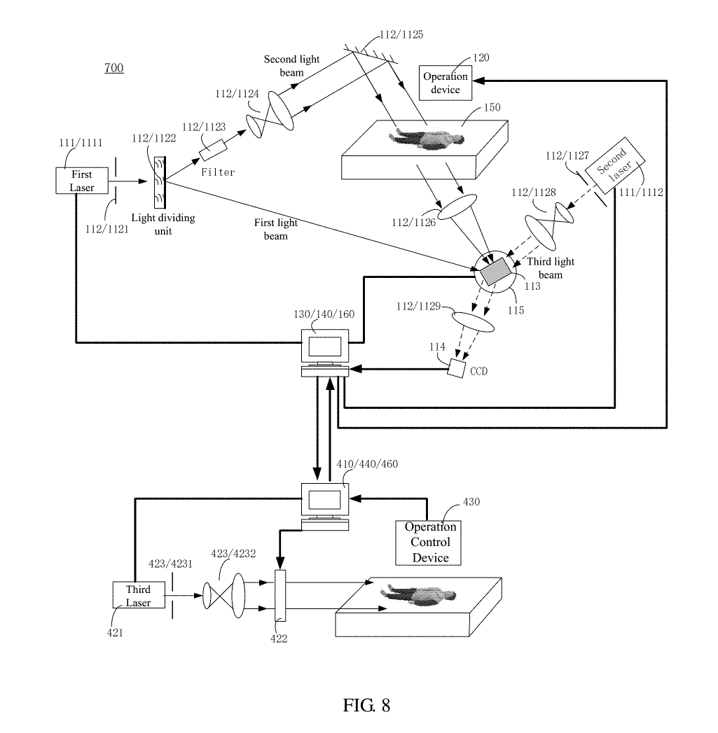

The fifth embodiment of the present disclosure further provides a telemedicine system. FIG. 7 is a schematic block diagram of the telemedicine system provided by the present disclosure. FIG. 8 is a schematic structural view of the telemedicine system provided by the present disclosure. As illustrated in FIGS. 7 and 8, the telemedicine system 700 comprises the holotherapy equipment provided by the first embodiment of the present disclosure and the holotherapy control equipment provided by the third embodiment of the present disclosure. The telemedicine system 700, for instance, may achieve the functions such as remote diagnosis and remote surgery. But the embodiment of the present disclosure is not limited thereto.

In the telemedicine system 700, the holotherapy equipment 100 transmits the holographic image information including the operated object. After receiving the holographic image information including the operated object, the holotherapy control equipment generates an operation control signal according to the image information, and sends the operation control signal to the holotherapy equipment. The holotherapy equipment performs medical operation on the operated object according to the received operation control signal. No detailed description will be given here for the simplificity of the Description. The specific structures and functions may refer to the holotherapy equipment provided by the first embodiment of the present disclosure and the holotherapy control equipment provided by the third embodiment of the present disclosure.

The telemedicine system provided by the embodiment of the present disclosure realizes the telemedicine of the doctor by generating and transmitting the holographic image information including the operated object. As the holographic image can accurately and comprehensively reflect the patient's image, the accuracy of telemedicine can be improved and the operation error can be reduced.

What are described above is related to the illustrative embodiments of the disclosure only and not limitative to the scope of the disclosure. Obvious variations and replacement by any one of the skilled person in the art in the technical scope of the disclosure should be all covered in the scope of this disclosure. The scopes of the disclosure are defined by the accompanying claims.

The application claims priority to the Chinese patent application No. 201610232400.4, filed Apr. 14, 2016, the disclosure of which is incorporated herein by reference as part of the application.

1. Holographic operation equipment, comprising:
a holographic image generating device configured to generate holographic image information including an operated object;
an operation device configured to operate the operated object;
an image transmitting device configured to transmit the holographic image information; and
an information receiving device configured to receive a control signal and send the control signal to the operation device to control the operation of the operation device, wherein
the control signal is obtained according to the transmitted holographic image information.

2. The operation equipment according to claim 1, wherein the holographic image generation device includes: a light source component, an optical module, a holographic storage medium and an image acquisition means.

3. The operation equipment according to claim 2, wherein the light source component can emit a first light beam and a second light beam irradiating the operated object; the second light beam is led to the operated object by the optical module; light reflected or transmitted from the operated object is irradiated to the holographic storage medium after interfering with the first light beam, so as to store the holographic image information in the holographic storage medium;
the light source component also emits a third light beam; the third light beam irradiates the holographic storage medium to generate imaging beams emitted from the holographic storage medium; the imaging beams irradiate the image acquisition means; and the image acquisition means generates data corresponding to the holographic image information and sends the data to the image transmitting device.

4. The operation equipment according to claim 3, wherein the light source component includes a first laser; the optical module includes a light dividing unit; and an initial light beam emitted by the first laser is divided into the first light beam, the second light beam and the third light beam by the light dividing unit.

5. The operation equipment according to claim 3, wherein the light source component includes a first laser and a second laser; the optical module includes a light dividing unit; an initial light beam emitted by the first laser is divided into the first light beam and the second light beam by the light dividing unit; and the second laser emits the third light beam.

6. The operation equipment according to claim 2, wherein the holographic storage medium includes photorefractive crystals, photochromic materials or photopolymers.

7. The operation equipment according to claim 3, wherein the holographic storage medium is disposed on a shifter; and when the holographic operation equipment operates, the shifter allows different positions of the holographic storage medium to face light beams generated after the light reflected or transmitted from the operated object interferes with the first light beam.

8. The operation equipment according to claim 7, wherein the shifter is a revolving table.

9. The operation equipment according to claim 3, wherein the emission position of the third light beam may change around the holographic storage medium.

10. The operation equipment according to claim 2, wherein the image acquisition means includes a charge-coupled device (CCD) or complementary metal oxide semiconductor (CMOS) imaging unit.

11. The operation equipment according to claim 1, wherein the operation device includes a mechanical arm; and surgical instruments may be disposed on the mechanical arm.

12. The operation equipment according to claim 1, wherein the image transmitting device and the information receiving device are respectively in signal connection with an external device via Internet.

13-21. (canceled)

22. Holographic control equipment, comprising:
an image receiving device configured to receive holographic image information;
a holographic image reproduction device configured to reproduce a holographic image according to the holographic image information;
an operation control device configured to generate an operation control signal; and
an information output device configured to receive the operation control signal and output the operation control signal, wherein
the operation control signal is generated according to the holographic image.

23. The control equipment according to claim 22, wherein the image reproduction device includes:
a light source configured to emit reproduced beams;
a spatial light modulator configured to receive the holographic image information and convert the holographic image information into optical signals when irradiated by the reproduced beams; and
an imaging unit configured to reveal the optical signals as the holographic image.

24. The control equipment according to claim 22, wherein the light source includes lasers.

25. The control equipment according to claim 24, wherein the spatial light modulator includes a liquid crystal light valve, a micro-electro-mechanical system (MEMS) spatial light modulator, a digital micromirror device (DMD), photorefractive crystals or an acousto-optic modulator (AOM).

26. The control equipment according to claim 22, wherein the operation control device includes a touch element, an operating arm or a somatosensory control element.

27. A holographic control method, comprising:
receiving holographic image information;
reproducing a holographic image according to the holographic image information;
generating an operation control signal; and
receiving the operation control signal and outputting the operation control signal, wherein
the operation control signal is generated according to the holographic image.

28. The holographic control method according to claim 27, wherein the step of reproducing the holographic image according to the holographic image information includes:
emitting reproduced beams to a spatial light modulator;
allowing the spatial light modulator to receive the holographic image information and convert the holographic image information into optical signals when irradiated by the reproduced beams; and
revealing the optical signals as the holographic image.

29-31. (canceled)

32. A telemedicine system, comprising a holographic operation equipment and the holographic control equipment according to claim 22,
wherein the holographic operation equipment comprising:
a holographic image generating device configured to generate holographic image information including an operated object;
an operation device configured to operate the operated object;
an image transmitting device configured to transmit the holographic image information; and
an information receiving device configured to receive a control signal and send the control signal to the operation device to control the operation of the operation device, wherein
the control signal is obtained according to the transmitted holographic image information.
